(19) United States
(12) Patent Application Publication
(10) Pub. No.: US 2022/0265950 A1
(43) Pub. Date: Aug. 25, 2022

(54) Patient Airway Positioning Apparatus

(71) Applicant: Kerri Ahlf, Richmond, RI (US)

(72) Inventor: Kerri Ahlf, Richmond, RI (US)

(21) Appl. No.: 17/675,261

(22) Filed: Feb. 18, 2022

Related U.S. Application Data
(60) Provisional application No. 63/200,191, filed on Feb. 19, 2021.

(51) Int. Cl. A61M 16/06

(52) U.S. Cl. CPC A61M 16/0683; A61M 2210/1032

(57) ABSTRACT

A wearable apparatus designed to assist an anesthesia provider in maintaining a patent airway comprises an elastomeric mandible harness with projecting members, elastic straps and connecting rings. The mandible harness is designed to hold the patient in a “jaw-thrust” position during surgical procedures. The apparatus attaches to a standard operating-table frame.

### TECHNICAL FIELD

The present disclosure relates to patient airway positioning apparatuses for the head and neck which immobilize the head during medical procedures.

### BACKGROUND

During procedures involving anesthesia, a patient's airway may become occluded, causing apnea. MAC (Monitored Anesthesia Care) is a type of anesthesia in which a patient is breathing on their own, without intubation. Normally during MAC anesthesia, the anesthesia provider must keep at least one hand on the patient's mandible to keep the airway patent (unobstructed); otherwise, the patient's airway will obstruct, which causes oxygen deprivation.

Devices that maintain an open airway, including specialized cervical collars and operating-table devices that hold the head and neck in rigid position, can interfere with medical procedures; some devices can compromise the cervical spine or result in spinal-cord damage during fast-paced emergency procedures.

A patient's trachea may become blocked by the slackened lower mandible. A manual method of holding a patient's mandible in a position that keeps the trachea open while the patient is immobilized is known as a jaw-thrust maneuver. It involves manipulating a patient's mandible to keep the trachea open while performing a medical procedure. The technique is used on patients in a supine position to open the patient's trachea. In this maneuver, a practitioner uses their thumbs to push the posterior of the mandible forward and into an open-airway position. As the mandible is displaced forward, it pulls the tongue forward and prevents it from occluding the entrance to the trachea. This helps establish a patent airway. The procedure can be problematic as it requires the provider to attempt to attend to more than one task at one time.

Among the sections of the human mandible, the inferior border is oriented at its base, below the body and in front of the angle of the ramus. This disclosure refers to the upper (outer) and lower (inner) inferior borders of a patient's mandible.

### SUMMARY

The disclosed embodiment is a wearable apparatus designed to assist an anesthesia provider in maintaining a patent airway on a patient in supine position. This allows the provider to perform other necessary tasks such as drawing up and administering medications and documenting during surgery.

The embodiment is substantially comprised of a mandible harness, straps and connecting rings. The mandible harness is designed to hold the patient in a “jaw-thrust” position. In an example embodiment a harness is constructed of common hardware and straps that surround the patient's head about the mandible. The harness has two curvilinear wedge forms shaped to hold the patient's mandible forward to provide access to the trachea. The two wedge forms curve and are contoured to meet the upper and lower areas of the inferior border of a patient's mandible. An example wedge form has at its top end a relatively high incline, and at its bottom end a relatively lower incline, with a concavity between, into which the patient's mandible rests. (The area of the mandible that rests in the concavity of the wedge forms is generally referred to as the inferior border.) The wedge forms are mounted to a cradle that is a flexible surface configured to extend from one side of a person's mandible to the opposite side. One skilled in the art understands that such contoured pads that make up the wedge forms and cradle may be constructed of foam padding such as polyurethane foam or of castable-elastomeric materials, and may be made in various sizes to fit various anatomies.

A rigid bar enables clamping of the apparatus to the operating table by use of standard clamps found on operating tables.

An adjustable strap, attached to the back of the cradle, extends on two sides of the patient's mandible to attach to a standard operating-table frame structure, to which it is tethered by a standard loop and D-ring connection or other buckle mechanism known in the art. The strap's length is adjustable on two sides through the aforementioned buckling means. An elastic-band portion of the strap absorbs movement of the patient's body during a surgical procedure. This elasticity keeps the head and spine in alignment and immovably secured so as to avoid spinal-cord injury.

The apparatus's parts are interchangeable and can be sanitized between uses.

### BRIEF DESCRIPTION OF THE DRAWINGS

FIG. 1 is a perspective, front view of the embodiment.

FIG. 2 is a perspective, side view of the embodiment.

FIG. 3 is a rear, perspective and detail view of the embodiment

FIG. 4 is an orthographic, front view of the embodiment.

FIG. 5 is an orthographic view of the anatomy of a mandible.

### DESCRIPTION

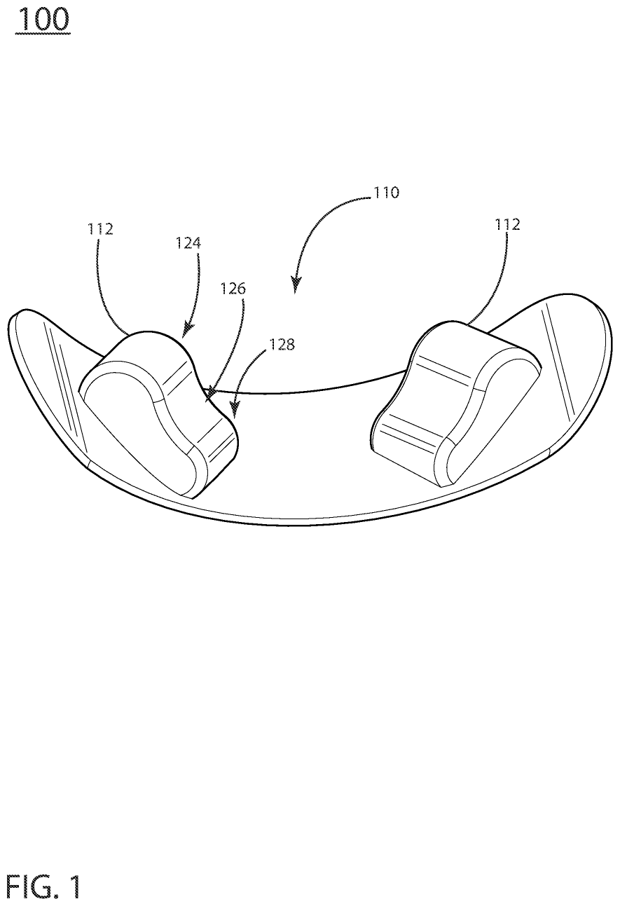

In FIG. 1 an example embodiment 100 shows the mandible cradle 110 of the apparatus. It holds a patient's head in a jaw-thrust position. Wedge forms 112 are affixed to the cradle 110 and are contoured to meet the upper and lower inferior borders of a patient's mandible. The illustrated wedge forms have at their top ends a relatively high incline 124, and at their bottom ends a relatively lower incline 128, with a concavity 126 between, into which the patient's mandible rests. One skilled in the art understands that variously sized wedge forms are used to fit various patients.

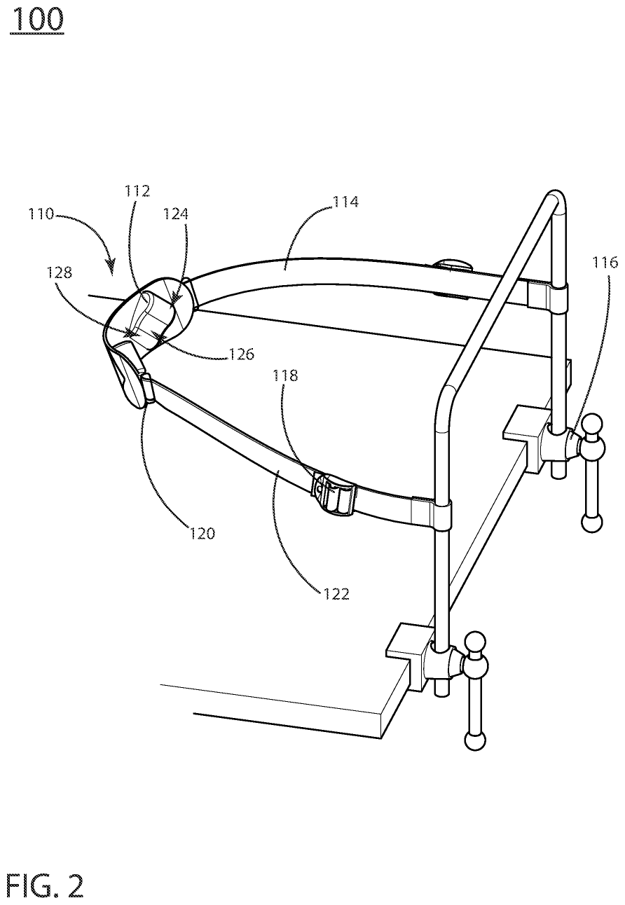

FIGS. 2, 3 and 4 are numbered alike. FIG. 2, 100 shows the example embodiment attached to a standard operating-table frame structure 116. The mandible cradle 110 holds the patient's head in jaw-thrust position, with the attached strap 114 at the back of the cradle extending to reach a standard operating-table frame structure 116, to which it is tethered by a standard loop and D-ring connection 118 known in the art. The strap's length is adjustable on two sides through a buckle mechanism 120 known in the art. An elastic-band portion of the strap 122 is designed to absorb the movement of the patient's body during a surgical procedure. This keeps the head and spine in alignment and immovably secured to avoid spinal-cord injury.

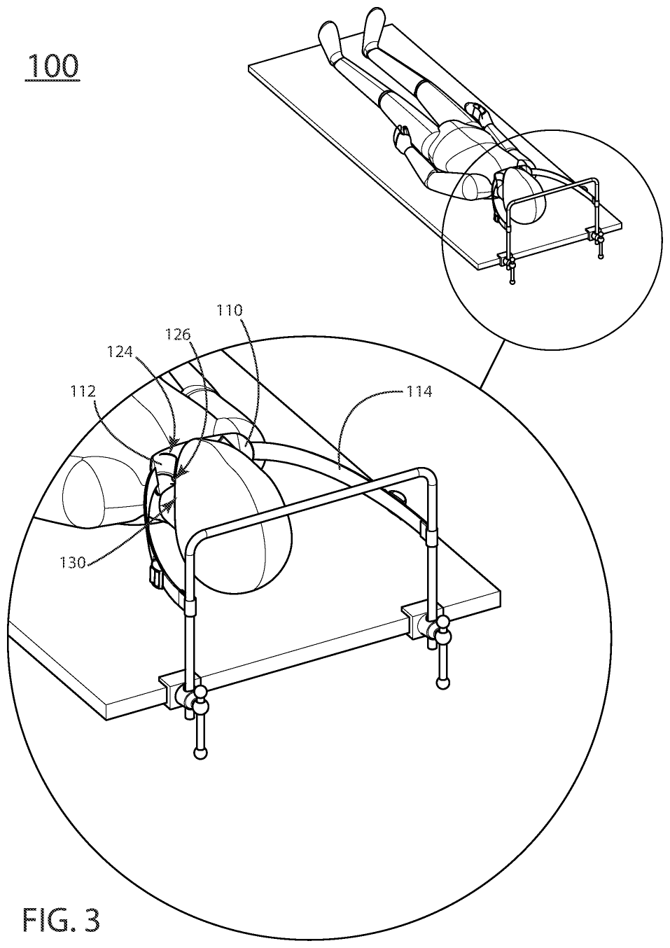

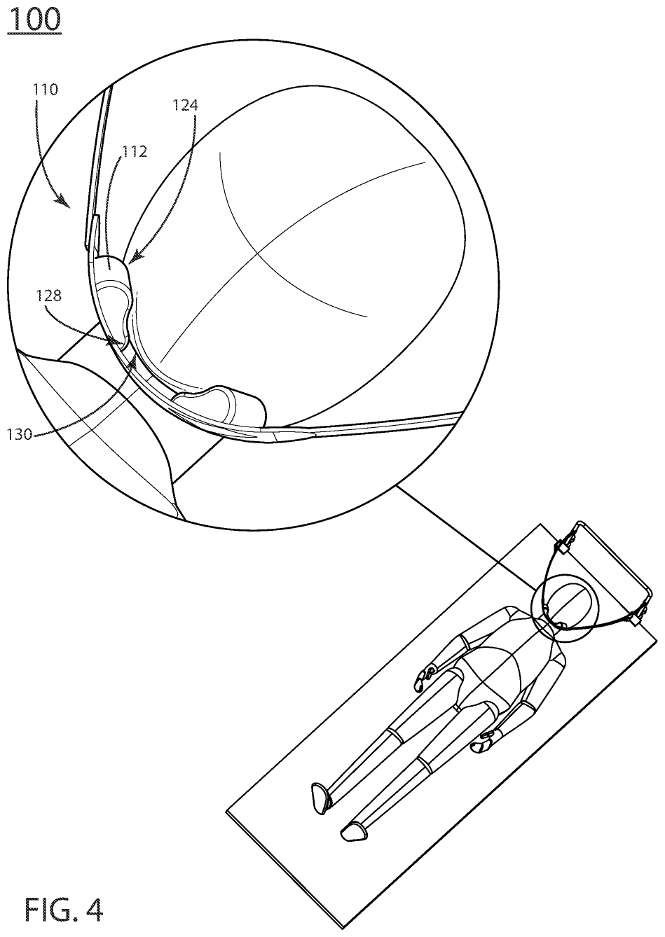

FIGS. 3 and 4 show the apparatus in use. Numbers are the same as those in FIG. 2. Jaw-thrust position is achieved by adjusting straps 114. In the example embodiment, the mandible cradle 110 extends around the base of the patient's mandible 130. The two wedge forms 112 are contoured to meet the upper and lower inferior borders of a patient's mandible. The illustrated wedge forms have at their top ends a relatively high incline 124, and at their bottom ends a relatively lower incline 128, with a concavity 126 (FIG. 2) between, into which the patient's mandible rests.

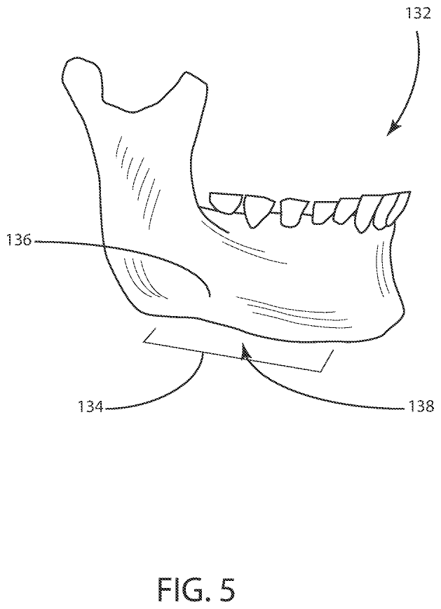

FIG. 5 shows the area of a mandible 132 generally known as the inferior border 134, 136 indicates the upper, or outer part of the inferior border, and 138 indicates the lower, or inner part of the inferior border.

These embodiments are exemplary and should not be construed as limiting.

1. An apparatus for positioning a patient's mandible comprising:
a cradle;
at least one wedge form fixedly engaged with said cradle and configured to fit against the patient's mandible; wherein
the patient's mandible is held fast by the at least one wedge form in combination with the cradle to maintain the patient's airway open.

2. The apparatus of claim 1, the at least one wedge form further comprising:
a first section of wedge form with a relatively higher incline, which transitions to a concave section, which transitions to a second, relatively lower-inclined section of wedge form; wherein
the patient's mandible resides in said concave portion, while said first section of wedge form resides on the upper part of the inferior border of the patient's mandible, and said second, relatively lower-inclined section resides on the lower part of the inferior border of the mandible, so that the patient's mandible is cradled.

3. The apparatus of claim 2 further comprising:
at least one strap configured to affix at a first end to a provided operating-table frame structure; and configured to affix at a second end to said cradle; and
at least a portion of said at least one strap being elastic; wherein
the at least one strap having at least a portion being elastic, holds the cradle and the at least one wedge form against the patient's mandible while the strap provides a force in the direction toward said operating table frame structure.

4. An apparatus for positioning a patient's mandible to maintain the patient's airway comprising:
a cradle;
at least one wedge form fixedly engaged with said cradle and configured to fit against the patient's mandible; and
an adjustable strap fixedly engaged with the cradle; and
the adjustable strap having at least an elastic portion; and
the adjustable strap removably engaged to a provided operating-table frame; wherein
the at least one wedge form configured to fit against the patient's mandible cradles the mandible to hold it in a “jaw-thrust” position; and said strap at least one elastic section stretches with the patient's movement.
